(19) United States
(12) Patent Application Publication
(10) Pub. No.: US 2002/0058299 A1
(43) Pub. Date: May 16, 2002

(54) Mycologic genera specific selective agar and method of manufacture

(76) Inventors: Richard A. Billups, Marietta, GA (US); Paul S. Warden, Johnson, VT (US); Kristen S. Fallon, Essex, VT (US); Jennifer E. Parent, Essex Jct., VT (US)

Correspondence Address: Law Offices Eric R. Benson, Esq., Eric R. Benson, Champlain Station, Box 65238, Burlington, VT, 05406-5238, US

(21) Appl. No.: 10/011,038

(22) Filed: Nov. 13, 2001

Related U.S. Application Data
(60) Provisional application No. 60/249,147, filed on Nov. 16, 2000.

(51) Int. Cl. C12Q 1/34; C12Q 1/04; C12N 1/18

(52) U.S. Cl. 435/34; 435/255.7

(57) ABSTRACT

A new, novel and useful mycologic genera specific selective agar method of manufacture and use. One of the benefits that is derived from this agar is the ability to quickly and easily selectively grow user selected mycologic genera which include the development of characteristic colony morphology, coloration and spore arrangement and texture. The growth medium or agar contains user specified anti-fungal and anti-bacterial compounds that inhibit the growth and development of competitive fungi and bacteria.

### TECHNICAL FIELD OF THE INVENTION

The present invention relates to mycologic genera specific selective agar for the recovery, identification and quantification of user specified fungi from environmental samples. The agar or growth medium restricts the growth and development of competitive fungi and bacteria and allows the unrestricted growth and development of characteristic colony morphology and coloration for the user selected genera.

For example both of the fungi Stachybotrys chartarum and Memnoneilla echinata have been implicated in such maladies as “Sick Building Syndrome” (SBS) and pediatric pulmonary hemosiderosis. SBS are non-specific maladies that appear to be related to the time spent inside a building. Stachybotrys chartarum has also been linked to adverse neurological effects including memory loss. The American Industrial Hygiene Association has named Stachybotrys chartarum as one of four organisms that have no acceptable levels in indoor air. This invention relates to the growth and identification in medium from environmental samples of user selected fungi including pathogenic fungi such as Stachybotrys chartarum and Memnoniella echinata.

### BACKGROUND ART

The use of selective agars for growth of microorganisms is well known in the prior art. Microbiologists have historically used specialized, selective agars for the preferential recovery of various types of bacteria. Examples include mEndo agar, used for the recovery of a broad group of bacteria known as coliform, Pseudomonas Isolation Agar (PIA), for the selective recovery of bacteria in the genus Pseudomonas (RNA Group 2) and Buffered Charcoal Yeast Extract (BCYE) for the recovery of Legionella species.

Selective agars have been less frequently employed for fungi and those that have been used typically target broad groups, such as xerophyllic fungi (those tolerant of low water availability), rather than specific genera. For example in U.S. Pat. No. 2,437,766 (Elmer Stevenson, et al.—Mar. 6, 1948) the patent discloses and with six claims teaches a method of culturing fungi of the genera Penicillium, Fusarium and Rhizoctonia. The method teaches the use of a selective media (agar) specifically designed to inhibit only contaminating bacterial growth with the use of varying concentrations of 2,4-dichlorophenoxyacetic acid and the alkali metal salts of that acid.

It is also common practice to add anti-fungal compounds to bacterial agar and conversely, anti-bacterial compounds to fungal agar, to reduce or eliminate undesired (non-target species) growth. U.S. Pat. No. 5,849,516 (Wu et al.—Dec. 15, 1998) discloses and claims a semiselective medium for detecting the seed-borne fungus Alternaria brassicicola and method of use of that media. This patent uses the semi-selective composition which includes: galactose, calcium nitrate, dipotassium hydrogen phosphate, magnesium sulfate, agar, benomyl, and chloramphenicol. Despite this prior art there has been no prior use of such growth media or agars where both anti-bacterial and anti-fungal compounds are added to the fungal agar with the express intent of restricting the growth of bacteria and specific aggressive types of fungi, respectively for the detection and growth of Stachybotrys chartarum and Memnoniella echinata.

While the prior art discloses that various media have been previously suggested as suitable for the recovery of Stachybotrys, including Oatmeal Agar, Corn Meal Agar and Cellulose Agar, it will be noticed that none of the prior art cited disclose a media or agar that necessarily exclude other fungi or allow optimal development of diagnostic colony morphology of user selected fungi. One of the difficulties in the recovery of Stachybotrys chartarum is that it is a relatively slow growing fungi and is often outcompeted by more aggressive fungi in the relatively limited environment of laboratory cultures. Another difficulty is rapid, positive identification of Stachybotrys chartarum which on many agars fails to develop characteristic coloration or spores.

The culturing and growth of Stachybotrys chartarum and Memnoniella echinata in Potato Dextrose Agar (PDA) and other agars are well known in the prior art. These methods of growths however, are not designed to selectively culture these organisms from an environmental sample, rather they are used to grow purified inoculations of the single organism. For example in U.S. Pat. No. 4,217,345 (Shiuohara et al.—Aug. 12, 1980) it discloses and claims the therapeutic agent 3-0(β-D-Glucuronopyranosyl)-soyasapogenol B. This patent discloses the use of Stachybotrys in various cultures including PDA to produce the drug or pharmaceutical agent, 3-0(β-Glucuronopyranosyl)-soyasapogenol B. This patent is of interest because it teaches the growth of Stachybotrys in PDA culture. Also in U.S. Pat. No. 4,229,466 (Miyazaki et al.—Oct. 21, 1980) the patent discloses and claims a group of pharmaceutical compounds generally derived from sesquiterpene. The claims also include a claim (6) to a method of treating nephritis with these derivatives. Similar to Shinohara U.S. Pat. No. 4,217,345 above this patent also discloses the use of Stachybotrys in various cultures including PDA to produce the drug or pharmaceutical agent, sesquiterpene derivatives. In U.S. Pat. No. 4,254,225 (Tamura et al.—Mar. 3, 1981) the patent discloses and claims a glucoamylase enzyme and method for producing the same with the use of Stachybotrys subsimplex. Similar to Shinohara U.S. Pat. No. 4,217,345 and Miyazaki et al. U.S. Pat. No. 4,229,466 above this patent discloses the use of Stachybotrys to produce a chemical compound grown in cultures including PDA. Lastly in U.S. Pat. No. 4,981,980 (Giocobbe et al.—Jan. 1, 1991) discloses and claims a drug for treating manic depression which is an inositol monophosphate phosphatase inhibitor that is extracted from the fermentation of a nutrient medium by the Hypomycetes fungus of the genera of Stachybotrys and Memnoniella.

### DISCLOSURE OF THE INVENTION

In view of the foregoing limitations inherent in the known types of mycologic genera specific selective agars now present in the prior art, the present invention provides an agar and method of manufacture and use that has been designed to allow the user to selectively culture from an environmental sample mycologic genera specific organisms, which are improvements that are patently distinct over similar growth media which may already be patented or commercially available. As such, the general purpose of the present invention, which will be described subsequently in greater detail, is to provide a new, novel and useful user selected mycologic genera specific selective agars and method of manufacture and use. There are many additional novel features of this invention directed to solving problems not addressed in the prior art as follows:

Promotes more rapid growth of Stachlybotrys than other selective or general media, allowing quantitative determination of S. chartarum concentrations within one week.

Allows development of characteristic (diagnostic) Stachybotrys colony morphology, coloration and spore structures—significantly more so than other media.

Restricts the growth of commonly recovered aggressive fungi that would otherwise outcompete Stachybotrys for the limited nutrients and available space in the laboratory culture environment.

Restricts the growth of bacteria able to grow on fungal agar.

Does not inhibit Stachybotrys growth, therefore yielding accurate representation of the sample environment.

To attain this, the present invention generally comprises an agar, typically a potato dextrose agar (PDA) to which is introduced a particular mixture of nutrients and chemical compounds to inhibit the growth of bacteriological and mycological organisms other than the particular mycologic genera specific selected organism to be detected and cultured.

There has thus been outlined, rather broadly, the more important features of the invention in order that the detailed description thereof that follows may be better understood, and in order that the present contribution to the art may be better appreciated. There are additional features of the invention that will be described hereinafter and which will form the subject matter of the claims that will be made once the full application is filed.

In this respect, before explaining at least one embodiment of the invention in detail, it is to be understood that the invention, method and apparatus, is not limited in its application to the details of construction and to the arrangements of the components set forth in the following description. The invention, method and apparatus, is capable of other embodiments and of being practiced and carried out in various ways. Also, it is to be understood that the phraseology and terminology employed herein are for the purpose of description and should not be regarded as limiting in any way the scope of this invention or claims made herein.

As such, those skilled in the art will appreciate that the conception, upon which this disclosure is based, may readily be utilize as a basis for the designing of other is structures, methods and systems for carrying out the several purposes of the present invention. It is important, therefore, that the claims be regarded as including such equivalent constructions and methods insofar as they do not depart from the spirit and scope of the present invention.

Further, the purpose of the foregoing abstract is to enable the U.S. Patent and Trademark Office and the public generally, and especially the scientists, engineers, and practitioners in the art who are not familiar with patent or legal terms or phraseology, to determine quickly from a cursory inspection, the nature and essence of the technical disclosure of the application. The abstract is neither intended to define the invention of the application, which is measured by the claims, nor is it intended to be limiting as to the scope of the invention in any way.

It is therefore an object of the present invention to provide a new, novel and useful mycologic genera specific selective agar and method of manufacture and use which has many of the advantages of the mycologic genera specific selective agars mentioned heretofore and many novel features that result in a mycologic genera specific selective agar and method of manufacture and use which is not anticipated, rendered obvious, suggested, or even implied by any of the prior art mycologic genera specific selective agars and methods of manufacture, either alone or in any combination thereof.

It is another object of the present invention to provide a mycologic genera specific selective agar which may be easily and efficiently manufactured, taught and marketed.

It is a further object of the present invention to provide a mycologic genera specific selective agar which is of a durable and reliable construction.

An even further object of the present invention is to provide a mycologic genera specific selective agar which is susceptible of a low cost of manufacture with regard to both materials and labor, and which accordingly is then susceptible of low prices of sale to the consuming public, thereby making such mycologic genera specific selective agar economically available to the buying public.

Still yet another object of the present invention is to provide a mycologic genera specific selective agar which provides in the apparatuses of the prior art some of the advantages thereof, while simultaneously overcoming some of the disadvantages normally associated therewith.

These together with other objects of the invention, along with the various features of novelty which characterize the invention, shall be pointed out with particularity in the claims annexed to and forming a part of this disclosure upon the filing of the full application. For a better understanding of the invention, its operating advantages and the specific objects attained by its uses, reference should be had to the accompanying descriptive matter in which there is explained the preferred embodiments of the invention.

### BEST MODE FOR CARRYING OUT THE INVENTION

In order to be successful in culturing Stachybotrys from environmental samples, Aspergillus flavus, Aspergillus fumigatus, Aspergillus vervicolor, Cladosporium species, Penicillium species, in addition to other fungi and bacteria, need to be suppressed. While conceptually simple, the difficulty is to determine which base media, in combination with which one or more antibiotics, at what concentrations, incubated at what temperature regimes, for how long, would achieve inhibition of competitors—without restricting Stachybotrys. Extensive experimental trials empirically determined the Minimum Inhibitory Concentration (MIC) levels of various compounds that would effectively suppress aggressive fungi without restricting Stachybotrys development. Through this empirical effort there has been developed a proprietary formula for a selective agar (PDA+) based on commercially available base media and additional ingredients for the specific recovery of Stachybotrys chartarum.

PDA+ allows identification of Stachybotrys in less than one week, typically in three to five days (without any subcultures or additional media). In contrast other media such as CMA may allow rapid Stachybotrys growth, but without characteristic coloration and only in the absence of competitive microorganisms. Still other media, such as cellulose agar, allows development of characteristic coloration and generally limits some competitors, but may require 2-3 weeks or more for Stachybotrys growth.

In addition to restricting competitive growth and allowing the rapid development of characteristic morphology, PDA+ recovers accurate concentrations of Stachybotrys. The data from controlled trials using seeded samples (without competitive fungi) analyzed with PDA+ and other media known to support the growth of Stachybotrys, indicate that the antimicrobial compounds in PDA+ do not suppress Stachybotrys species. Repeated trials on environmental samples, including aerosol, surface and bulk samples, have demonstrated the superiority of PDA+ to all prior art listed above. Prior to the development of PDA+, no single media had been demonstrated to recover accurate concentrations of Stachybotrys from environmental samples containing competitive fungi and bacteria while also allowing identification within one week.

### EXAMPLE

The preferred embodiment of PDA+ is manufactured by mixing in three parts the ingredients as follow:

(1) Potato Dextrose Agar (PDA)—Version: 200.0 grams (g) Infusion from Potatoes, 20.0 grams (g) Dextrose, 15.0 grams (g) Agar. Prepared from bulk powder in accordance with manufacturer's instructions.

(2) Miconazole (antifungal compound added to base media at 1.6 micrograms (μg) per milliliter (ml) of PDA+, the optimum concentration within the functional range of 1.0 to 3.2 μg/ml).

(3) Chloramphenicol (antibacterial compound added to base media at 0.1 grams (g) per liter (1) of PDA+).

Samples to be analyzed on PDA+ may be collected as aerosol surface or bulk samples and may be streaked, spread plated or plated using membrane filters according to standard microbiological techniques described elsewhere. Samples are incubated for 7 days at temperatures of 25° Centigrade (C) and 34° C. Starting at Day 3, samples are examined daily for Stachybotrys, and colonies are enumerated as soon as reliable identifications can be made. Identification of Stachybotrys is made by gross examination of colony morphology and coloration plus microscopic examination of conidial structure and spore, arrangement and texture, Competitive fungi that are able to grow on PDA+ are so restricted in development that they do not inhibit Stachybotrys growth, nor do they impede the investigator from identifying target colonies. One skilled in the art can relatively easily differentiate Stachybotrys colonies from competitive types of fungi on PDA+, allowing accurate enumeration of recovered colonies.

Therefore, investigators are able to recover, identify and enumerate viable Stachybotrys from environmental samples more rapidly and with greater sensitivity with the present invention than with conventional methods.

As to a further discussion of the manner of usage and operation of the present invention, the same should be apparent from the above description. Accordingly, no further discussion relating to the manner of usage and operation will be provided.

With respect to the above description then, it is to be realized that the optimum dimensional relationships and formulae for the parts of the invention, to include variations in size, quantity of materials, shape, form, function and manner of operation, assembly and use, are deemed readily apparent and obvious to one skilled in the art, and all equivalent relationships to those described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

Having described our invention, We claim:

1. A mycologic genera specific selective agar comprising:
an agar capable of supporting the growth of fungi;
the agar further comprising one or more antibacterial compound and one or more antifungal compound.

2. The mycologic genera specific selective agar of claim 1 wherein the agar is further comprised of infusion from potato, an agar medium and dextrose.

3. The mycologic genera specific selective agar of claim 1 wherein the antibacterial compound is chloramphenicol.

4. The mycologic genera specific selective agar of claim 1 wherein the antifungal compound is miconazole.

5. The mycologic genera specific selective agar of claim 2 wherein the antibacterial compound is chloramphenicol.

6. The mycologic genera specific selective agar of claim 2 wherein the antifungal compound is miconazole.

7. A mycologic genera specific selective agar comprising:
an agar;
the agar further comprising an infusion from potato, an agar medium, dextrose, chloramphenicol and miconazole.

8. The mycologic genera specific selective agar of claim 7 wherein the agar is further comprised of 200 grams of the infusion from potato, 15 grams of an agar medium and 20 grams of the dextrose.

9. The mycologic genera specific selective agar of claim 7 wherein the concentration of the chloramphenicol is 0.1 grams of the chloranphenicol per liter of the agar.

10. The mycologic genera specific selective agar of claim 7 wherein the concentration of the miconazole is 1.0 to 3.2 micrograms of the miconazole per milliliter of the agar.

11. The mycologic genera specific selective agar of claim 8 wherein the concentration of the chloramphenicol is 0.1 grams of the chloramphenicol per liter of the agar.

12. The mycologic genera specific selective agar of claim 8 wherein the concentration of the miconazole is 1.0 to 3.2 micrograms of the miconazole per milliliter of the agar.

13. A mycologic genera specific selective agar comprising:
an agar;
the agar ftuther comprising 200 grams of infusion from potato, 15 grams of an agar medium, 20 grams of dextrose, 0.1 grams of chloramphenicol per liter of the agar and 1.0 to 3.2 micrograms of miconazole per milliliter of the agar.

14. A method of manufacturing the mycologic genera specific selective agar of claim 1 comprising the steps of:
identifying a genera of fungi to be detected if present in a sample that is to be tested;
selecting the agar containing nutrients that will promote the growth of the identified genera of fungi;
selecting one or more of the antibacterial compounds that will retard or prevent the growth of bacteria in the sample when exposed thereto and such that the identified genera of fungi's growth would not be prevented thereby;
selecting one or more of the antifungal compounds that will retard or prevent the growth of competitive fungi in the sample when exposed thereto and such that the identified genera of fungi's growth would not be prevented thereby; and
mixing the agar with one or more of the selected antibacterial compounds and one or more of the selected antifungal compounds such that the concentration of the selected antibacterial compounds and the selected antifungal compounds retard or prevent the growth of bacteria and competitive fungi and such that the identified genera of fungi's growth would not be prevented thereby.

15. A method of using the mycologic genera specific selective agar of claim 1 comprising the steps of:
identifying a genera of fungi to be detected if present in a sample that is to be tested;
selecting the agar that contains one or more of the antibacterial compounds and one or more of the antifungal compounds which together retard or prevent the growth of bacteria and competitive fungi such that the identified genera of fungi's growth would not be prevented;
sterilizing the agar;
inoculating the sterilized agar with the sample or portion thereof using sterile technique;
incubating the inoculated agar under sterility, temperature and light conditions that maximize the growth potential of the identified genera of fungi to be detected in the sample if present; and
examining periodically the incubated, inoculated agar for evidence of growth of the identified genera of fungi to be detected in the sample if present.
